(19) United States
(12) Patent Application Publication
(10) Pub. No.: US 2008/0021083 A1
(43) Pub. Date: Jan. 24, 2008

(54) 4-Methylpyrazole Formulations for Inhibiting Ethanol Intolerance

(76) Inventor: Thomas E. Daley, San Mateo, CA (US)

Correspondence Address: JONES DAY, 222 EAST 41ST ST, NEW YORK, NY, 10017, US

(21) Appl. No.: 10/591,735

(22) PCT Filed: Mar. 3, 2005
(86) PCT No.: PCT/US05/07273

§ 371 Date: Jul. 23, 2007

Related U.S. Application Data
(60) Provisional application No. 60/550,261, filed on Mar. 3, 2004.
(60) Provisional application No. 60/642,007, filed on Jan. 6, 2005.

(51) Int. Cl. A61K 31/415; A61P 25/32; C07D 231/12

(52) U.S. Cl. 514/406; 548/373.1

(57) ABSTRACT

Methods, articles of manufacture and compositions are provided useful for the prevention or amelioration of a symptom of acetaldehyde accumulation or ethanol intolerance in a subject with reduced or absent reduced or absent aldehyde dehydrogenase subtype 2 (ALDH2) activity wherein about 1 mg/kg to about 4 mg/kg 4-methylpyrazole, or an equivalent mass of a 4-MP salt, are to be orally administered to the subject.

### RELATED APPLICATIONS

This application claims the benefit of U.S. provisional application No. 60/642,007, filed Jan. 6, 2005, and U.S. provisional application No. 60/550,261, filed Mar. 3, 3004, which are incorporated herein by reference in their entireties.

### 1. TECHNICAL FIELD

The present application relates to formulations comprising 4-methylpyrazole (4-MP), or physiologically acceptable salts thereof, and a physiologically acceptable excipient, and their use to prevent or ameliorate ethanol intolerance in a subject with reduced or absent aldehyde dehydrogenase subtype 2 (ALDH2) activity.

### 2. BACKGROUND OF THE INVENTION

Ethanol consumed by a person is removed from the bloodstream, in large part, in a two-step pathway in which ethanol is oxidized by alcohol dehydrogenase (ADH) to acetaldehyde, a toxin that is in turn quickly metabolized into acetic acid by aldehyde dehydrogenase subtype 2 (ALDH2), a mitochondrial liver enzyme. A significant portion of the human population is “ALDH2 deficient” and carries a variant ALDH2 allele that produces an ALDH2 enzyme with reduced activity that results in a 40%-90% reduction in the rate of acetaldehyde removal. While various health disorders for those individuals in the larger human population, including liver cirrhosis and hepatocellular carcinoma, can occur after long-term use of ethanol, in ALDH2 deficient individuals, these disorders can result from ingestion of ethanol at relatively low doses over a shorter period of time. See Ohira (1996) Alcohol Clin. Exp. Res. 20:378a-382a; Takeshita et al. (2000) Cancer Lett. 149:69-76. The experience of ethanol consumption in ALDH2 deficient individuals is marked by facial flushing, accelerated heart rate, and a subjective sense of sickness rather than the euphoria and/or relaxation that typically accompanies ethanol consumption. See Ward et al. (1994) Alcohol and Alcoholism 29:433-438.

A recognized ADH inhibitor, 4-methylpyrazole (also known as fomepizole or 4-MP), has been approved by the U.S. Food and Drug Administration for the treatment of ethylene glycol or methanol poisoning. See, e.g., Scalley et al. (2002) American Family Physician 66:807-812. In poison victims, ADH metabolizes ethylene glycol or methanol into toxic by-products such as oxalates and glycolates, and dosing regimens of 4-MP are predicated on the need to completely inhibit ADH, to the extent possible, in order to prevent severe damage to multiple organ systems by the toxic by-products produced by ADH. As a treatment for ethylene glycol or methanol poisoning, the administration of 4-MP generally requires intravenous infusion under the supervision of a doctor in relatively large doses.

Although the elevated acetaldehyde concentrations produced from ADH activity on ethanol carry significant health risks, these risks are not as acute nor as imminent as in the ethylene glycol or methanol poisoning contexts for which 4-MP is currently administered. Persistence of 4-MP in a person's blood stream for time periods measured in days—as is the case with currently practiced dosing of 4-MP—is highly undesirable for subjects with ALDH2 deficiency who wish to consume ethanol, as they typically wish the effects of elevated blood ethanol levels to be relatively temporary, lasting from minutes to several hours. In addition, high doses of 4-MP inhibit ADH activity to an extent that human subjects treated with 4-MP can have much higher blood ethanol concentrations than when not treated with 4-MP. As such, the removal of ethanol from the blood stream at the 4-MP doses described in the literature is too slow to allow safe recreational use of ethanol.

Moreover, administration of high doses, e.g., 50 mg/kg body mass and greater, of 4-MP itself has been reported to cause side effects similar to ALDH2 deficiency including flushing, headache and nausea. See Jacobsen et al. (1988) Alcoholism: Clinical and Experimental Research 12:516-522. The dosages taught in the literature also have an undesirable side effect profile with chronic use. While appropriate in the acute care context, preclinical data in mice indicate the danger of up to 30% shrinkage of the testicular mass after several weeks of use.

What remains to be determined are solutions to address the adverse consequences of acetaldehyde accumulation as a result of ethanol consumption in that portion of the human population having a defective version of ALDH2. Preferably, such solutions will minimally impact the ethanol elimination rate and thereby avoid the consequence of having relatively lengthy periods of time during which the subject is under the influence of ethanol, and avoid the undesirable side-effects associated with higher doses of 4-MP.

### 3. SUMMARY OF THE INVENTION

In one aspect, the present invention provides methods and compositions useful for preventing or ameliorating a symptom of acetaldehyde accumulation, or ethanol intolerance, in a subject with reduced or absent ALDH2 activity.

In certain aspects, methods are provided for preventing or ameliorating ethanol intolerance in a subject with reduced or absent aldehyde dehydrogenase subtype 2 (ALDH2) activity comprising administering 4-MP to the subject.

A symptom of acetaldehyde accumulation in a subject can be, for example, selected from the group consisting of flushing, elevated heart rate, palpitations, hypotension, nausea, dizziness, and headache.

In certain aspects, methods are provided for preventing a disease associated with the long term use of ethanol in a subject with reduced or absent ALDH2 activity. Diseases associated with the long term use of ethanol can include, for example and without limitation, liver cirrhosis and cancer, including hepatocellular carcinoma, mouth cancer, stomach cancer, and esophageal cancer.

In some embodiments, the methods provided comprise administering to the subject about 1 mg to about 4 mg 4-methylpyrazole (4-MP), or the equivalent mass of 4-MP in a physiologically acceptable salt form, per kilogram of the subject's body mass.

In certain embodiments, MP-4 is orally administered.

In some embodiments, 4-MP is orally administered before the subject consumes ethanol.

In some embodiments, 4-MP is orally administered about two hours to about fifteen minutes before the subject consumes ethanol.

In other embodiments, 4-MP is orally administered concurrently with the subject's consumption of ethanol or after the subject has consumed ethanol.

In certain embodiments, the percent reduction in the subject's ethanol elimination rate is no more than about 10% in comparison to the ethanol elimination rate of a subject not administered 4-MP.

In some embodiments, the method can comprise administering an effective amount of 4-MP that reduces acetaldehyde accumulation by about 25% to about 60% as compared to a subject not administered 4-MP.

In some embodiments, the method can comprise administering an amount of 4-MP or a physiological acceptable salt of 4-MP effective to reduce or inhibit ethanol-oxidizing activity of alcohol dehydrogenase in the subject.

In certain embodiments, an effective amount of a hydrochloride salt of 4-MP is administered.

In other aspects, the present invention provides articles of manufacture. In certain embodiments an article of manufacture can comprise packaging material and a composition comprising 4-MP, or a physiologically acceptable salt thereof, and a physiologically acceptable excipient, suitable for oral administration to a subject.

In some embodiments, the form of the composition is liquid.

In other embodiments, the form of the composition is a tablet.

In certain embodiments, where the article of manufacture comprises a composition further comprising 4-MP in a tablet form, the tablet comprises about 85 milligrams of 4-MP.

In some embodiments, the article of manufacture can also comprise printed instructions regarding the use or administration of the composition. In certain embodiments, the printed instructions suggest a dosing regimen for the prevention or amelioration of a symptom of acetaldehyde accumulation accompanying ethanol consumption in a subject.

In other aspects, the present invention provides a composition comprising 4-MP, or a physiologically acceptable salt thereof, and a physiologically acceptable excipient, suitable for oral administration to a subject.

In other aspects, the present invention provides methods for identifying agents with therapeutic potential for the prevention or amelioration of symptoms associated with ALDH2 deficiency. The agent is considered a potential therapeutic agent if ADH enzyme inhibition is noted in vitro using techniques as described below.

### 4. DESCRIPTION OF FIGURES

FIG. 1. Graph of milligrams 4-MP per kilogram body mass administered to human subjects versus percent reduction in ethanol elimination rate. Linear least squares regression was used to fit a line to data obtained from the sources cited in Section 7.

### 5. TERMINOLOGY

As used herein, “about” indicates a range of +/−10%. For example, “about 4 mg 4-MP” means a range of from 3.6 mg to 4.4 mg 4-MP.

As used herein, the term “dose” or “dosage” refers the amount of 4-MP that an individual takes or is administered at one time. The term “unit dosage form” refers to a physically discrete unit, such as a capsule, tablet or volume of liquid, suitable as a unitary dosage for a human subject. Each unit contains a predetermined quantity of 4-MP that was discovered as a result of this invention to produce the desired pharmacokinetic profile which yields the desired therapeutic effect. The dosage unit is composed of 4-MP in association with at least one pharmaceutically acceptable carrier, salt, excipient, or combination thereof. By way of example, an 170 mg 4-MP dose refers to amount of 4-MP a person can take at one time, where the dose can be divided into two 85 mg dosage units, for example, two 85 mg 4-MP tablets.

The phrase “symptom of acetaldehyde accumulation accompanying ethanol consumption,” as used herein refers to any symptom experienced by subjects with reduced or absent ALDH2 activity when consuming ethanol. Symptoms can include, but are not limited to, flushing, elevated heart rate, palpitations, hypotension, nausea, dizziness, and headache.

The phrase “subject with a reduced or absent aldehyde dehydrogenase subtype 2 (ALDH2) activity” refers to a subject that is a homozygous or heterozygous carrier of the variant ALDH2*2 allele of the ALDH2 gene as described in Goedde et al. (1992) Hum. Genet. 88:344-346 and Xiao et al. (1995) J. Clin. Invest. 96:2180-2186, which are incorporated herein by reference in their entireties, or to a subject that expresses any variant ALDH2 enzyme that exhibits less activity than the normal ALDH2 enzyme as determined by the aldehyde dehydrogenase activity assay described in Xiao et al. (1995) J. Clin. Invest. 96:2180-2186.

As used herein, “ethanol intolerance,” refers to a condition in which a subject experiences a symptom of acetaldehyde accumulation accompanying ethanol consumption.

As used herein, “ethanol elimination rate” refers to the reduction in ethanol concentration in a subject's bloodstream over time after the subject has ingested ethanol. Typically, an ethanol elimination rate can be expressed in terms of millimole ethanol/kilogram subject body mass/hour. Techniques for blood sampling and analysis of ethanol levels in blood are well known to those of skill in the art. See, e.g., Inoue et al. (1984) Alcoholism: Clinical and Experimental Research 8:319-322, incorporated herein by reference in its entirety. A “percent change in ethanol elimination rate,” can be calculated as follows:

$$\begin{matrix}Percent Change in \\ Et OH Elimination\end{matrix} = ( 1 - \frac{\begin{matrix}Subject ' s Et OH Elimination \\ Rate After Taking 4 \text{-} MP\end{matrix}}{\begin{matrix}Subject ' s Et OH Elimination \\ Rate Prior to Taking MP \text{-} 4\end{matrix}} ) \times 100$$

where EtOH represents ethanol, and a number for a percent change in ethanol elimination that is less than 100 is a reduction in the percent change in EtOH elimination. Blood ethanol levels can also be calculated, for example, based on algorithms utilizing the amount of ethanol consumed by a subject, the subject's body mass, and time period since the consumption of ethanol, or, as another example, blood ethanol levels can be extrapolated from analysis of a subject's breath, and the like, as known to those of skill in the art.

As used herein, “acetaldehyde accumulation” refers to the production of acetaldehyde in a subject that has consumed ethanol. Techniques for blood sampling and analysis of acetaldehyde levels in blood are well known to those of skill in the art. See, e.g., Inoue et al. (1984) Alcoholism: Clinical and Experimental Research 8:319-322; Stowell (1979) Clin. Chim. Acta. 98:201-5, each incorporated herein by reference in its entirety. Maximal concentrations of acetaldehyde accumulation typically follow fifteen minutes to one hour following ethanol consumption in a subject with reduced or absent ALDH2 activity. Where a “percent change in acetaldehyde accumulation” is used herein, this will be understood to mean the change in the maximal concentrations of acetaldehyde in a subject with reduced or absent ALDH2 activity, that can be calculated as follows:

$$\begin{matrix}Percent Change in \\ Acetaldehyde \\ Accumulation\end{matrix} = ( 1 - \frac{\begin{matrix}Max . Acetaldehyde Conc . \\ After Taking 4 \text{-} MP\end{matrix}}{\begin{matrix}Max . Acetaldehyde Conc . \\ Prior to Taking MP \text{-} 4\end{matrix}} ) \times 100$$

where a number for a percent change in acetaldehyde accumulation that is less than 100 is a reduction in the percent change in acetaldehyde accumulation. Blood acetaldehyde concentrations can also be extrapolated from analysis of a subject's breath, or from measurable physiological changes in other parameters, such as heart rate or flushing, and the like, as known to those of skill in the art.

The term “physiologically acceptable salt,” as used herein, refers to the relatively nontoxic, inorganic and organic acid addition salts of compounds of the invention.

As defined herein, where the mass of 4-MP is specified, for example, “2 mg 4-MP,” that amount refers to the equivalent mass of 4-MP in its free base form. Thus, for example, if 2 mg 4-MP in a given salt form is to be administered in a method disclosed herein, those of skill in the art can make the necessary conversion using the molecular masses of the salt form of 4-MP and of the free base form of 4-MP to determine the actual mass of that salt form of 4-MP necessary to obtain the equivalent mass of 2 mg 4-MP in its free base form. As another example, if 2 mg 4-MP in a free base form is to be administered in a method disclosed herein, then no conversion is necessary.

### 6. DETAILED DESCRIPTION OF THE INVENTION

The present invention provides compositions and methods useful for ameliorating the severity of, or preventing, an adverse physiological symptom associated with acetaldehyde accumulation accompanying ethanol consumption in a subject with reduced or absent aldehyde dehydrogenase subtype 2 (ALDH2) activity.

Reduced or absent aldehyde dehydrogenase subtype 2 (ALDH2) activity are evident in a person that consumes low or moderate amounts of ethanol, most typically by cutaneous flushing. ALDH2 deficiency can be a result of, for example, a genetic mutation in the ALDH2 gene. See Xiao et al. (1995) J. Clin. Invest. 96:2180-2186. It is generally believed that the acetaldehydemia, or accumulation of acetaldehyde, is responsible for the symptoms exhibited in people with ALDH2 deficiency after consuming ethanol. Adverse symptoms associated with acetaldehyde accumulation can include, for example, flushing, elevated heart rate, nausea, dizziness, headache, and the like.

As explained below, the methods provided comprise the administration of 4-MP or a physiologically acceptable salt of 4-MP. Without intending to be bound by any particular theory, it is believed that 4-MP acts to inhibit alcohol dehydrogenase (ADH) to reduce the accumulation of acetaldehyde production that results from the consumption of ethanol. As disclosed herein, relatively small doses of 4-MP, for example, about 1 mg/kg to about 4 mg/kg, administered to a subject with reduced or absent ALDH2 activity, can significantly increase the comfort level of the subject by preventing or ameliorating the symptoms of acetaldehyde accumulation with minimal reduction of the subject's ethanol elimination rate.

### 6.1. Methods to Prevent or Ameliorate a Symptom of Acetaldehyde Accumulation

In certain aspects, the present invention provides methods for preventing or ameliorating a symptom of acetaldehyde accumulation or ethanol intolerance in a subject with reduced or absent ALDH2 activity. In some embodiments, the method can comprise administering about 1 mg to about 4 mg 4-methylpyrazole (4-MP) per kilogram of a subject's body mass, to the subject.

In certain embodiments, the compound for use in the methods is the free base of 4-MP. In other embodiments, a physiologically acceptable salt of 4-MP can be used in the methods. In some embodiments, a 4-MP hydrochloride salt can be used in the methods described herein.

4-Methylpyrazole (4-MP, also known as fomepizole) is commercially available from chemical suppliers, including, for example, Sigma Aldrich (St. Louis, Mo.), and can also be synthesized easily in commercially viable quantities of pharmaceutical grade.

4-MP can be administered alone or in combination with other substances or active agents. In some embodiments, a composition comprising 4-MP and other ingredients, as described below, is administered.

4-MP can be administered according to any technique known to those of skill in the art. In certain embodiments, 4-MP can be delivered transdermally. In preferable embodiments, the subject can self-administer 4-MP to himself or herself. In preferable embodiments, 4-MP can be administered orally. When orally administered, 4-MP can be in a solid form, for example, as in a powder, tablet, capsule and the like, or in a liquid form.

In certain embodiments, the amount of 4-MP administered can be between about 0.1 mg/kg to about 4 mg/kg. In some embodiments, between about 1 mg/kg to about 4 mg/kg 4-MP can be administered. As will be understood by those of skill in the art, the amounts of 4-MP to be administered, as described herein, are based on the body mass of the subject, expressed in kilograms. In some embodiments, about 0.1 mg/kg, about 0.5 mg/kg, about 1.5 mg/kg, about 2 mg/kg, about 2.5 mg/kg, about 3 mg/kg, about 3.5 mg/kg, or about 4 mg/kg of 4-MP are administered to the subject having reduced or absent ALDH2 activity. In certain embodiments, the amount of 4-MP administered can be in the range between 0.1 mg/kg to 3 mg/kg, in the range between 0.5 mg/kg to 2 mg/kg, or in the range between 2 mg/kg to 4 mg/kg.

In certain embodiments, the amount of 4-MP, or physiologically acceptable salt thereof, administered can be effective to reduce or inhibit the ethanol-oxidizing activity of alcohol dehydrogenase in the subject.

In certain embodiments, 4-MP can be administered before the subject has consumed ethanol. In some embodiments, 4-MP can be administered about one minute, about fifteen minutes, or about one hour before the subject consumes ethanol. In some embodiments, 4-MP can be orally administered about two hours to about fifteen minutes before the subject consumes ethanol.

In certain embodiments, 4-MP can be administered concurrent with the consumption of ethanol. In certain embodiments, 4-MP can be administered immediately before or after the consumption of ethanol. In some embodiments, 4-MP can be administered to a subject after the subject has consumed ethanol.

As suggested above, it is particularly advantageous to minimize the peak concentrations of acetaldehyde in the blood of subjects with reduced or absent ALDH2 activity that occur during the consumption of ethanol, without a concomitant reduction in the rate of ethanol elimination. With the doses of 4-MP contemplated in the instant methods, that is, between about 1 mg/kg to about 4 mg/kg, the percent reduction in ethanol elimination rate can be negligibly or minimally impacted as discussed below.

In certain embodiments, methods are provided comprising the administration of 4-MP wherein a percent reduction in ethanol elimination ranges from about 0%, about 1%, about 2%, about 3%, about 4%, about 5% or about 6% to no more than about 10%. For example, if a subject not treated with 4-MP that has an ethanol elimination rate of 2.50 mmol/kg/hr, and has an ethanol elimination rate of 2.30 mmol/kg/hr when administered with 4-MP, than the percent reduction in ethanol elimination is 8%.

In some embodiments, methods are provided that can have a percent reduction in the subject's ethanol elimination rate ranging from no reduction or 1-2% reduction in the ethanol elimination rate to less than about 7%, about 8%, about 9%, or about 10% reduction in the subject's rate of ethanol elimination. In some embodiments, the methods provided result in a reduction of ethanol elimination between about 5% to about 10%. In some embodiments, the percent reduction in the subject's ethanol elimination rate is no more than about 10% in comparison to the ethanol elimination rate of the subject not treated with 4-MP.

With the doses of 4-MP contemplated in the instant methods, the percent reduction in peak blood acetaldehyde concentrations can be reduced in a subject having reduced or absent ALDH2 activity.

In certain embodiments, the methods provided can reduce acetaldehyde accumulation by about 50% to about 60% in a subject with reduced or absent ALDH2 activity as compared to when 4-MP is not administered to the subject. In certain embodiments, the peak acetaldehyde accumulation can be effectively eliminated or reduced by about 95%, 90%, 85%, 80%, 75%, 70%, 65%, 60%, 55%, 50%, 45%, 40%, 35%, 30%, 25%, 20%, 15%, 10% or about 5%.

It will be noted that for the subject population with reduce or absent ALDH2 activity, the instant methods, if not eliminating one or more symptoms of acetaldehyde accumulation altogether, should reduce the severity of one or more symptoms by a substantial degree for large percentage of the patient population, and will therefore be a useful method for treatment of a broad spectrum of this subject population.

In certain embodiments, the methods provided prevent or ameliorate a symptom of acetaldehyde accumulation in a subject selected from the group consisting of flushing, elevated heart rate, palpitations, hypotension, nausea, dizziness, and headache.

Although any person having, or suspected of having, a reduced or absent ALDH2 activity may be treated with 4-MP as described herein, certain subpopulations may be identified that would especially benefit. For example, the invention encompasses preferred methods wherein 4-MP is used on subjects who have a history of robust cutaneous flushing of the face when consuming alcohol, and/or are known to carry a the variant ALDH2 allele that encodes a glutamate to lysine substitution at position 487 of the mitochondrial aldehyde dehydrogenase enzyme.

In certain aspects, the present invention provides methods for preventing a disease associated with the long term use of ethanol in a subject with reduced or absent ALDH2 activity. In general, diseases associated with the long term use of ethanol include, for example and without limitation, liver cirrhosis and cancer, for example, hepatocellular carcinoma, mouth cancer, stomach cancer, and esophageal cancer. In some embodiments, the method can comprise administering about 1 mg to about 4 mg 4-MP per kilogram of a subject's body mass, to the subject. In certain embodiments, 4-MP can be administered before, during or after the subject consumes ethanol. In some embodiments, 4-MP is administered orally. In some embodiments, a physiologically acceptable salt of 4-MP is administered.

In certain preferable embodiments of methods provided for preventing a disease associated with the long term use of ethanol in a subject, 4-MP is administered prior to the consumption of ethanol by the subject. In some embodiments, 4-MP can be administered within about two hours before the subject consumes ethanol.

### 6.2. Articles of Manufacture

In certain aspects, the present invention provides articles of manufacture useful for preventing or ameliorating a symptom of acetaldehyde accumulation or ethanol intolerance in a subject with reduced or absent ALDH2 activity.

In certain embodiments, an article of manufacture comprises packaging material, and a composition comprising 4-MP, or a physiologically acceptable salt thereof, and a physiologically acceptable excipient, suitable for oral administration to a subject.

In certain embodiments, the form of the composition is liquid.

In some embodiments, the form of the composition is a solid selected from the group consisting of powder, tablet and capsule.

In certain embodiments, the composition in the article of manufacture comprises a unit dosage form of 4-MP or a physiologically acceptable salt thereof. In some embodiments, the unit dosage form comprises about 85 milligrams of 4-MP or an equivalent mass in a salt form thereof.

In some embodiments, the article of manufacture comprises a label or printed instructions regarding the use or administration of the composition. Typically, printed instruction can suggest a dosing regimen for the prevention or amelioration of a symptom of acetaldehyde accumulation accompanying ethanol consumption in a subject.

In certain embodiments, the printed instructions direct the subject to orally ingest a predetermined number of tablets according to the following table:

| Subject's | | |
|---|---|---|
| body mass | | No. of tablets to ingest |
| 36-46 | kg | 1 |
| 46-66 | kg | 2 |
| 66-86 | kg | 3 |
| 86-106 | kg | 4 |
| 106-126 | kg | 5. |

In certain embodiments, the printed instructions can suggest a dosing regiment for the prevention or amelioration of a symptom of acetaldehyde accumulation in a subject selected from the group consisting of flushing, elevated heart rate, palpitations, hypotension, nausea, dizziness, and headache.

### 6.3. Compositions

In certain aspects, the present invention provides compositions useful for preventing or ameliorating a symptom of acetaldehyde accumulation or ethanol intolerance in a subject with reduced or absent ALDH2 activity. Compositions of the present invention can be used in the manufacture of medicaments or formulations for the prevention or amelioration of a symptom of acetaldehyde accumulation or ethanol intolerance in a subject with reduced or absent ALDH2 activity.

In certain aspects, the present invention provides compositions useful for preventing a disease associated with the long term use of ethanol in a subject with reduced or absent ALDH2 activity. Compositions of the present invention can be used in the manufacture of medicaments or formulations for the prevention of a disease associated with the long term use of ethanol in a subject with reduced or absent ALDH2 activity. In certain embodiments, the disease associated with the long term use of ethanol is selected from the group consisting of liver cirrhosis, cancer, hepatocellular carcinoma, mouth cancer, stomach cancer, and esophageal cancer.

In certain embodiments, a composition is provided comprising 4-MP, or a physiologically acceptable salt thereof, and a physiologically acceptable excipient or diluent.

The compositions can be administered orally or transdermally. Compositions may take the form of powders, tablets, lozenges, granules, capsules, pills, ampoules, syrups, or fluids.

In certain embodiments, the composition can comprise 4-MP, or a salt thereof, in combination with one or more other active agents. Additional active agents can include, for example, a vitamin, anti-oxidant, an anti-inflammatory agent including, for example, aspirin, an nonsteroid anti-inflammatory drug, an antihistamine drug, ibuprofen, and the like.

In general, the composition will be formulated to conveniently allow administration of between about 1 mg/kg to about 4 mg/kg of 4-MP, or a salt thereof, to a subject in need thereof. In one embodiment, the unit dose form can be about 85 mg 4-MP or an equivalent mass in a salt form thereof. Unless otherwise indicated, all weights of active ingredient are calculated for 4-MP and would be increased proportionately for its salts.

A physiologically acceptable excipient must be “acceptable” in the sense of being compatible with the other ingredients of the formulation and not injurious to the patient.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

In a specific embodiment, the unit dosage is provided as a composition that is a tablet composed of 4-MP, microcrystalline cellulose, colloidal silicon dioxide, and magnesium stearate.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention may be formulated as an ointment, cream, suspension, lotion, powder, solution, past, gel, spray, aerosol or oil. Alternatively, a formulation may comprise a patch or a dressing such as a bandage or adhesive plaster impregnated with active ingredients and optionally one or more excipients or diluents.

It should be understood that in addition to the ingredients particularly mentioned above, the formulations of this invention may include other agents conventional in the art having regard to the type of formulation in question, for example, those suitable of oral administration may include such further agents as sweeteners, thickeners and flavoring agents.

### 7. EXAMPLES

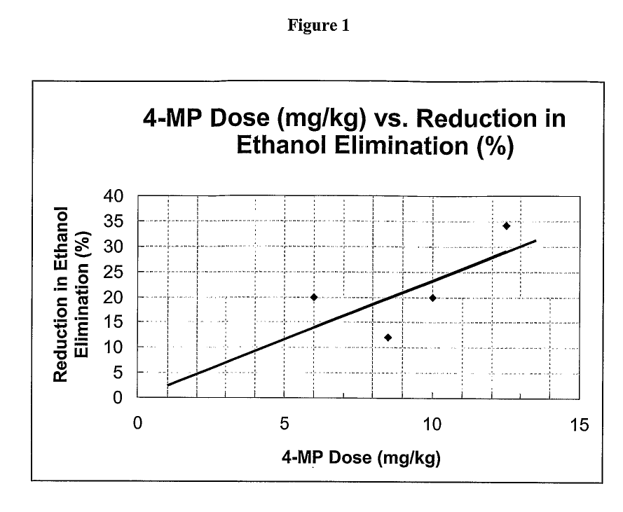

Without intending to be bound by any particular theory of operation, it is believed that when 4-MP in doses of about 4 mg/kg or less are administered to a human subject, the reduction in the elimination of ethanol consumed by the subject will be less than about 10%. FIG. 1 provides a graph of data representing amounts of 4-MP per kilogram body weight administered to human subjects versus observed percent reduction in ethanol elimination rates were obtained from the following sources and the averages determined as indicated in parenthesis: Lindros et al. (1981) Alcoholism: Clinical and Experimental Research 5: 528-530 (6 mg 4-MP; 20% reduction in EtOH elimination); Inoue et al. (1984) Alcoholism: Clinical and Experimental Research 8: 319-322 (10 mg 4-MP; 20% reduction in EtOH elimination); Inoue et al. (1985) Japan. J. Pharmacol. 38: 43-48 (8.5 mg 4-MP; 12% reduction in EtOH elimination); Sarkola et al. (2002) Alcoholism: Clinical and Experimental Research 26: 239-245 (12.5 mg 4-MP; 34% reduction in EtOH elimination). The data was plotted and linear least squares regression was used to fit a line to the data. The plot indicates that for doses of 4 mg/kg 4-MP and less, the ethanol elimination rate will be minimally impacted, i.e., that the reduction in ethanol elimination will be less than about 10%.

Exemplary administration of 4-MP to a human subject: 4-MP in its free base, liquid form is mixed with orange juice to make a 0.5% (w/v) 4-MP solution. The 4-MP may be stored in a container with an associated dispensing cup with markings indicating various amounts of solution to be used for different body masses of people to whom the 4-MP will be administered. For a person with a body mass of about 75 kg with reduced or absent ALDH2 activity who will be drinking alcohol, about 60 milliliters of the 4-MP is poured into the dispensing cup and the person with reduced or absent ALDH2 can drink the 4-MP solution from the cup in the minutes or hours prior to drinking alcohol.

Although the foregoing invention has been described in some detail by way of illustration and example for purposes of clarity of understanding, it will be apparent to those skilled in the art that certain changes and modifications will be practiced. Therefore, the description and examples should not be construed as limiting the scope of the invention, which is delineated by the appended claims.

It is claimed:

1. A method for preventing or ameliorating a symptom of ethanol intolerance in a subject with reduced or absent aldehyde dehydrogenase subtype 2 (ALDH2) activity comprising orally administering to the subject about 1 mg to about 4 mg 4-methylpyrazole (4-MP) per kilogram of the subject's body mass.

2. The method of claim 1, wherein 4-MP is administered in a free base form.

3. The method of claim 1, wherein 4-MP is administered in a physiologically acceptable salt form.

4. The method of claim 1, wherein 4-MP is orally administered before the subject consumes ethanol.

5. The method of claim 4, wherein 4-MP is orally administered about one hour to about fifteen minutes before the subject consumes ethanol.

6. The method of claim 1, wherein 4-MP is orally administered concurrently with the subject's consumption of ethanol or after the subject has consumed ethanol.

7. The method of claim 1, wherein the percent reduction in the subject's ethanol elimination rate is no more than about 10% in comparison to the ethanol elimination rate of a subject not administered 4-MP.

8. A method of preventing or reducing a symptom associated with acetaldehyde accumulation accompanying ethanol consumption in a subject with reduced or absent aldehyde dehydrogenase subtype 2 (ALDH2) activity comprising administering an effective amount of 4-MP that reduces acetaldehyde accumulation by about 50% to about 60% as compared to a subject not administered 4-MP.

9. The method of claim 8, wherein the subject with reduced or absent ALDH2 activity exhibits a percent reduction in ethanol elimination rate that is no more than about 10% in comparison to the ethanol elimination rate of a subject not administered 4-MP.

10. A method of ameliorating a symptom of acetaldehyde accumulation accompanying ethanol consumption in a subject with reduced or absent aldehyde dehydrogenase subtype 2 (ALDH2) activity comprising administering an amount of 4-MP or a physiologically acceptable salt of 4-MP effective to reduce or inhibit ethanol-oxidizing activity of alcohol dehydrogenase in the subject.

11. The method of claim 8 or 10, wherein a symptom of acetaldehyde accumulation in the subject with reduced or absent ALDH2 activity is selected from the group consisting of flushing, elevated heart rate, palpitations, hypotension, nausea, dizziness, and headache.

12. The method of claim 10 wherein an effective amount of a hydrochloride salt of 4-MP is administered.

13. The method of claim 10 wherein about 1 milligram to about 4 milligrams of 4-MP per kilogram of subject body mass is administered.

14. An article of manufacture comprising packaging material, and a composition comprising 4-methylpyrazole (4-MP), or a physiologically acceptable salt thereof, and a physiologically acceptable excipient, suitable for oral administration to a subject.

15. The article of manufacture of claim 14 wherein the form of the composition is liquid.

16. The article of manufacture of claim 14 wherein the form of the composition is a tablet.

17. The article of manufacture of claim 16 wherein the tablet comprises about 85 milligrams of 4-MP.

18. The article of manufacture of claim 16, further comprising printed instructions regarding the use or administration of the composition.

19. The article of manufacture of claim 18 wherein the printed instructions suggest a dosing regimen for the prevention or amelioration of a symptom of acetaldehyde accumulation accompanying ethanol consumption in a subject.

20. The article of manufacture of claim 19 wherein the printed instructions direct the subject to orally ingest a predetermined number of tablets according to the following table:

| Subject's | | |
|---|---|---|
| body mass | | No. of tablets to ingest |
| 36-46 | kg | 1 |
| 46-66 | kg | 2 |
| 66-86 | kg | 3 |
| 86-106 | kg | 4 |
| 106-126 | kg | 5. |
